(19) United States
(12) Patent Application Publication
(10) Pub. No.: US 2024/0100303 A1
(43) Pub. Date: Mar. 28, 2024

(54) FLEXIBLE LOCKABLE MICROCATHETER AND GUIDEWIRE CO-AXIAL SYSTEM

(71) Applicant: Boston Scientific Medical Device Limited, Galway (IE)

(72) Inventors: Prateek Gupta, Gurgaon (IN); Jahnavi Konduru, New Delhi (IN); Ishank Jain, New Delhi (IN); Anshul Chabra, Delhi (IN); Rahul Jain, Meerut (IN)

(73) Assignee: Boston Scientific Medical Device Limited, Galway (IE)

(21) Appl. No.: 18/475,073

(22) Filed: Sep. 26, 2023

Related U.S. Application Data
(60) Provisional application No. 63/410,071, filed on Sep. 26, 2022.

(51) Int. Cl. A61M 25/01; A61M 25/00; A61M 25/09; A61M 99/00

(52) U.S. Cl. CPC A61M 25/0113; A61M 25/0043; A61M 25/0097; A61M 25/0905; A61M 99/00; A61M 2025/0042; A61M 2025/09125

(57) ABSTRACT

A catheter system which provides adjustability of the length of the guidewire extending distally from the distal tip of the catheter and/or allows the guidewire to be secured relative to the catheter hub to allow the guidewire and the catheter to be steered simultaneously. The catheter system may include a catheter having an elongate shaft defining a catheter lumen, a hub assembly coupled to the proximal end of the elongate shaft and including a hub assembly lumen, a torque assembly releasably coupled to the hub assembly and including a torque assembly lumen, and a guidewire co-axially disposed within the catheter lumen, hub assembly lumen, and torque assembly lumen. In a first configuration the torque assembly may be configured to simultaneously torque the elongate shaft and the guidewire and in a second configuration the torque assembly may be configured to torque the guidewire independent of the elongate shaft.

### CROSS-REFERENCE TO RELATED APPLICATIONS

This application claims the benefit of priority under 35 U.S.C. § 119 of U.S. Provisional Application No. 63/410,071, filed Sep. 26, 2022, the entire disclosure of which is hereby incorporated by reference.

### TECHNOLOGY FIELD

The present application relates generally to a torquing device, and in particular, to a combined torquing and rotating hemostasis valve.

### BACKGROUND

Microcatheters may be used in selective interventional endovascular procedures to reach a target vessel for delivering various therapies. Microcatheters may be tracked over a guidewire to reach the target site. In general, guidewires may be steerable devices where a clinician uses an external torquing device to steer the guidewire to the target location. Typical preparation of the microcatheter and guidewire system may include flushing and dipping the microcatheter in saline water to activate the hydrophilic coating. The microcatheter may then be loaded over the guidewire and inserted into a guide catheter hub. During the procedure, the guidewire may be advanced in tandem with the microcatheter. A length of the guidewire extending distally from a distal end of the microcatheter may be adjusted as clinically required. The guidewire may be torqued by the clinician for effective and precise tracking of the microcatheter to the target site. In some cases, microcatheters may be available with a pre-loaded guidewire as a co-axial system. Generally, there may be two types of pre-assembled systems. Both the first and second pre-assembled systems may have limitations. For example, in a first pre-assembled system, the guidewire length extending distally from the distal tip of the microcatheter is limited and cannot be more than a certain predetermined amount. In a second preassembled system, the length of the guidewire extending distally from the distal tip of the microcatheter may be adjustable but the guidewire cannot be locked to the hemostasis hub to allow for simultaneous torquing of the microcatheter and the guidewire. There is an ongoing need to provide alternative medical devices as well as alternative methods for manufacturing and using medical devices.

### SUMMARY

This disclosure provides design, material, manufacturing methods, and use alternatives for medical devices.

In a first example, a catheter system may comprise a catheter having an elongate shaft extending from a proximal end to a distal end and defining a catheter lumen extending from the proximal end to the distal end, a hub assembly coupled to the proximal end of the elongate shaft, the hub assembly including a hub assembly lumen in fluid communication with the catheter lumen, a torque assembly releasably coupled to the hub assembly, the torque assembly including a torque assembly lumen in selective fluid communication with the hub assembly lumen, and a guidewire, the guidewire co-axially disposed within the catheter lumen, hub assembly lumen, and torque assembly lumen. In a first configuration the torque assembly may be configured to simultaneously torque the elongate shaft and the guidewire and in a second configuration the torque assembly may be configured to torque the guidewire independent of the elongate shaft.

Alternatively or additionally to any of the examples above, in another example, a length of the guidewire extending distally from the distal end of the elongate shaft may be adjustable.

Alternatively or additionally to any of the examples above, in another example, in the first configuration the torque assembly may be coupled to the hub assembly.

Alternatively or additionally to any of the examples above, in another example, in the second configuration the torque assembly may be uncoupled from the hub assembly.

Alternatively or additionally to any of the examples above, in another example, the torque assembly may comprise a rotatable valve, a main body, a collet, and an actuatable cap.

Alternatively or additionally to any of the examples above, in another example, the rotatable valve may comprise a first coupling portion rotatably coupled to a second coupling portion. The second coupling portion may be configured to rotate independently of the first coupling portion when the first coupling portion is held in a fixed position.

Alternatively or additionally to any of the examples above, in another example, when in the first configuration the torque assembly may be configured to torque the guidewire independently of the catheter shaft when the first coupling portion is held in the fixed position.

Alternatively or additionally to any of the examples above, in another example, a distal end region of the main body may be fixedly secured to the second coupling portion.

Alternatively or additionally to any of the examples above, in another example, the collet may be configured to selectively lock to the guidewire.

Alternatively or additionally to any of the examples above, in another example, the actuatable cap may be actuated to selectively lock the collet to the guidewire.

Alternatively or additionally to any of the examples above, in another example, the actuatable cap may be configured to selectively bias a proximal end region of the collet radially inwards to lock the collet to the guidewire.

Alternatively or additionally to any of the examples above, in another example, when the guidewire is unlocked from the collet, the guidewire may be actuatable independent of the torque assembly.

Alternatively or additionally to any of the examples above, in another example, the main body may comprise a flush port in fluid communication with the torque assembly lumen.

Alternatively or additionally to any of the examples above, in another example, the torque assembly may further comprise a hemostasis seal.

Alternatively or additionally to any of the examples above, in another example, the hemostasis seal may be configured to form a fluid tight seal between the guidewire and the main body of the torque assembly.

In another example, a catheter system may a catheter having an elongate shaft extending from a proximal end to a distal end and defining a catheter lumen extending from the proximal end to the distal end, a hub assembly coupled to the proximal end of the elongate shaft, the hub assembly including a hub assembly lumen in fluid communication with the catheter lumen, a torque assembly releasably coupled to the hub assembly, the torque assembly comprising a rotatable valve, a main body, a collet, and an actuatable cap and including a torque assembly lumen in selective fluid communication with the hub assembly lumen, and a guidewire, the guidewire co-axially disposed within the catheter lumen, hub assembly lumen, and torque assembly lumen and selectively secured relative to the torque assembly. In a first configuration the torque assembly may be configured to simultaneously torque the elongate shaft and the guidewire and in a second configuration the torque assembly may be configured to torque the guidewire independent of the elongate shaft. A length of the guidewire extending distally from the distal end of the elongate shaft may be adjustable.

Alternatively or additionally to any of the examples above, in another example, the torque assembly may further comprise a hemostasis seal.

Alternatively or additionally to any of the examples above, in another example, in the first configuration the torque assembly may be coupled to the hub assembly and in the second configuration the torque assembly may be uncoupled from the hub assembly.

Alternatively or additionally to any of the examples above, in another example, the rotatable valve may comprise a first coupling portion rotatably coupled to a second coupling portion. The second coupling portion may be configured to rotate independently of the first coupling portion when the first coupling portion is held in a fixed position and when in the first configuration the torque assembly may be configured to torque the guidewire independently of the catheter shaft when the first coupling portion is held in the fixed position.

In another example, a catheter system may comprise a catheter having an elongate shaft extending from a proximal end to a distal end and defining a catheter lumen extending from the proximal end to the distal end, a hub assembly coupled to the proximal end of the elongate shaft, the hub assembly including a hub assembly lumen in fluid communication with the catheter lumen, a torque assembly releasably coupled to the hub assembly, the torque assembly comprising a rotatable valve including a first coupling portion rotatably coupled to a second coupling portion, a main body, a collet, and an actuatable cap and including a torque assembly lumen in selective fluid communication with the hub assembly lumen, and a guidewire, the guidewire co-axially disposed within the catheter lumen, hub assembly lumen, and torque assembly lumen and selectively secured relative to the torque assembly. In a first configuration the torque assembly may be coupled to the hub assembly and may be configured to simultaneously torque the elongate shaft and the guidewire and in a second configuration the torque assembly may be uncoupled from the hub assembly and may be configured to torque the guidewire independent of the elongate shaft. A length of the guidewire extending distally from the distal end of the elongate shaft may be adjustable. When in the first configuration the torque assembly may be configured to torque the guidewire independently of the catheter shaft when the first coupling portion is held in the fixed position.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative catheter and torque assembly in a first configuration;

FIG. 2 is a cross-sectional view of the illustrative catheter and torque assembly taken at line 2-2 in FIG. 1; and

FIG. 3 is a perspective view of the illustrative catheter and torque assembly of FIG. 1 in a second configuration.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Microcatheters may be used in selective interventional endovascular procedures to reach a target vessel for delivering various therapies. Microcatheters may be tracked over a guidewire to reach the target site. In general, guidewires may be steerable devices where a clinician uses an external torquing device to steer the guidewire to the target location. Typical preparation of the microcatheter and guidewire system may include flushing and dipping the microcatheter in saline water to activate the hydrophilic coating. The microcatheter may then be loaded over the guidewire and inserted into a guide catheter hub. During the procedure, the guidewire may be advanced in tandem with the microcatheter. A length of the guidewire extending distally from a distal end of the microcatheter may be adjusted as clinically required. The guidewire may be torqued by the clinician for effective and precise tracking of the microcatheter to the target site. In some cases, microcatheters may be available with a pre-loaded guidewire as a co-axial system (e.g., the guidewire extending co-axially within the microcatheter). Generally, there may be two types of pre-assembled systems.

In a first pre-assembled system, the microcatheter and the guidewire hubs can be locked together. This system may have a fixed length of guidewire extending distally from a distal end of the microcatheter. This system may allow the guidewire to be tightly secured relative to the microcatheter as the fixed molded hub and/or torque device at the proximal end of the guidewire may be locked to the proximal hub of the microcatheter. However, this may not give the clinicians flexibility to advance or retract the guidewire ahead of the microcatheter distal tip as per their comfort and anatomy requirement during the procedure. For example, the length of guidewire extending distally from the distal end of the microcatheter may not be adjustable. In this setup, the fixed and/or molded hub on the guidewire proximal end may have a female Luer lock at its proximal end which allows the clinicians to flush the microcatheter lumen and guidewire simultaneously.

In a second pre-assembled system, a full-length guidewire may be pre-loaded through the microcatheter lumen in a manner which allows the guidewire to be adjustable and/or flexible relative to the microcatheter and without a fixed locking mechanism. This may require a separate secondary torquing device to effectively rotate the guidewire. Additionally, to flush the microcatheter before use, a separate hemostasis valve may be attached to the proximal hub of the microcatheter. This may be required because the torque device placed at the proximal end of the guidewire may not have any provision to flush the guidewire or the microcatheter. The same hemostasis valve may also be used during the procedure to prevent any blood loss and to help physicians lock the sheath of an embolic coil to facilitate easy transfer of a coil from the sheath to the microcatheter hub.

Both the first and second pre-assembled systems described herein may have limitations. For example, in the first pre-assembled system, the guidewire length extending distally from the distal tip of the microcatheter is limited and cannot be more than a certain predetermined amount. In the second preassembled system, the length of the guidewire extending distally from the distal tip of the microcatheter may be adjustable but the guidewire cannot be locked to the hemostasis hub to allow for simultaneous torquing of the microcatheter and the guidewire. The present disclosure is directed towards a microcatheter and guidewire system which provides adjustability of the length of the guidewire extending distally from the distal tip of the microcatheter as well as allows the guidewire to be secured relative to the microcatheter hub to allow the guidewire and the microcatheter to be steered simultaneously.

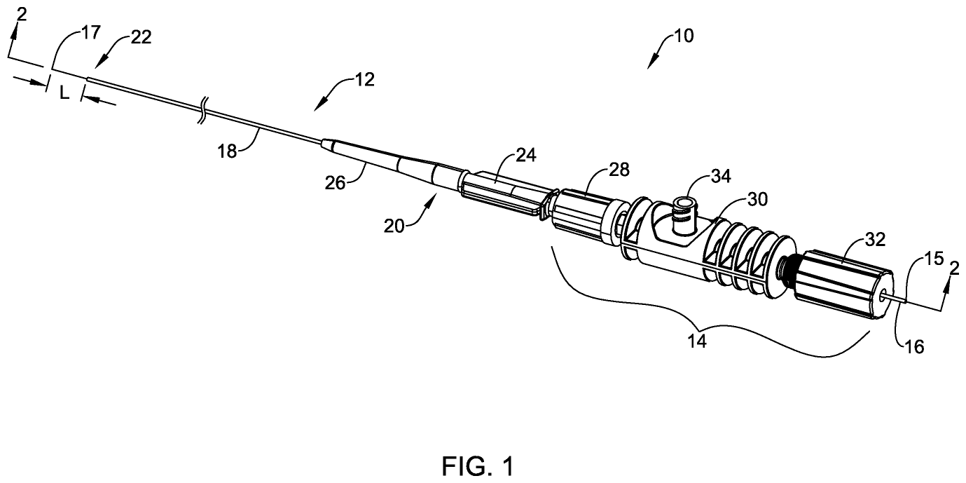

FIG. 1 is a perspective view of an illustrative catheter system 10 in accordance with an embodiment of the present disclosure. The system 10 may generally include a catheter 12, a torque assembly 14, and a guidewire 16. The catheter 12 can be one of a variety of different catheters. In some cases, the catheter 12 may be an intravascular catheter. Examples of some intravascular catheters include microcatheters, drug delivery catheters, diagnostic catheters, and guide catheters. FIG. 1 illustrates a microcatheter, but the disclosure is not limited to such. The catheter 12 can be manufactured using conventional techniques.

The catheter 12 can be sized in accordance with its intended use. For example, the catheter 12 can have a length that is in the range of about 50 to 200 centimeters and can have a diameter that is in the range of about 1.7 French (F), but can be as large as about 12 F for certain applications.

In the illustrated embodiment, the catheter 12 may include an elongate shaft 18 that has a proximal end 20 and a distal end 22. A hub assembly 24 can be connected to or disposed about the proximal end 20 of the elongate shaft 18. The hub assembly 24 may be secured to the catheter shaft 18 at the proximal end 20 of the shaft 18 using any suitable technique, for example, by adhesive, friction fitting, mechanically fitting, chemically bonding, thermally bonding, heat shrink materials, molding, casting, welding (e.g., resistance or laser welding), soldering, brazing, the use of an outer sleeve or polymer layer to bond or connect the components, or the like, or combinations thereof. In some embodiments, the distal end of the hub assembly 24 can be cast, molded or shaped onto the proximal end 20 of the shaft 18 such that it is connected to the proximal end 20. In other embodiments, the hub assembly 24 may be formed as a separate component and subsequently attached (e.g., adhered, press-fit, etc.) to the proximal end 20 of the catheter shaft 18. In some cases, the hub assembly 24 may include a strain relief 26, although this is not required. When so provided, the strain relief 26 may reduce kinking.

The guidewire 16 extends from a proximal end 15 to a distal end 17. The guidewire 16 may extend through a lumen of the torque assembly 14, through a lumen 48 of the hub assembly 24, and through a lumen of the elongate shaft 18. The distal end 17 of the guidewire 16 may extend distally beyond the distal end 22 of the elongate shaft 18. A length L of the guidewire 16 extending distally beyond the distal end 22 of the elongate shaft 18 may be increased or decreased, as desired. In some cases, the proximal end 15 of the guidewire 16 may extend proximally from a proximal end of the torque assembly 14, although this is not necessarily required.

The torque assembly 14 may include a rotating valve 28, a main body 30, and a movable cap 32 among other components. The torque assembly may be configured to receive guidewires and devices up to about 4F in diameter. As will be described in more detail herein, the movable cap 32 may be actuated to selectively secure the guidewire 16 relative to the torque assembly 14. For example, when the cap 32 is in a first configuration, the guidewire 16 may be translated proximally and/or distally along a longitudinal axis of the torque assembly 14. When the cap 32 is in a second configuration, axial translation of the guidewire 16 within the torque assembly 14 is precluded. In some cases, the torque assembly 14 may be axially translated with the cap 32 in the second configuration to axially translate the guidewire 16 relative to the catheter 12. A side flush port 34 may be provided in the main body 30 to flush the catheter 12 and/or guidewire 16. While not explicitly shown, the flush port 34 may include tubing to facilitate attachment of syringes to the flush port 34. In some embodiments, the flush port 34 may be omitted. In another example, the main body 30 may additionally include one or more hemostasis ports. The one or more hemostasis ports may provide options to the clinicians.

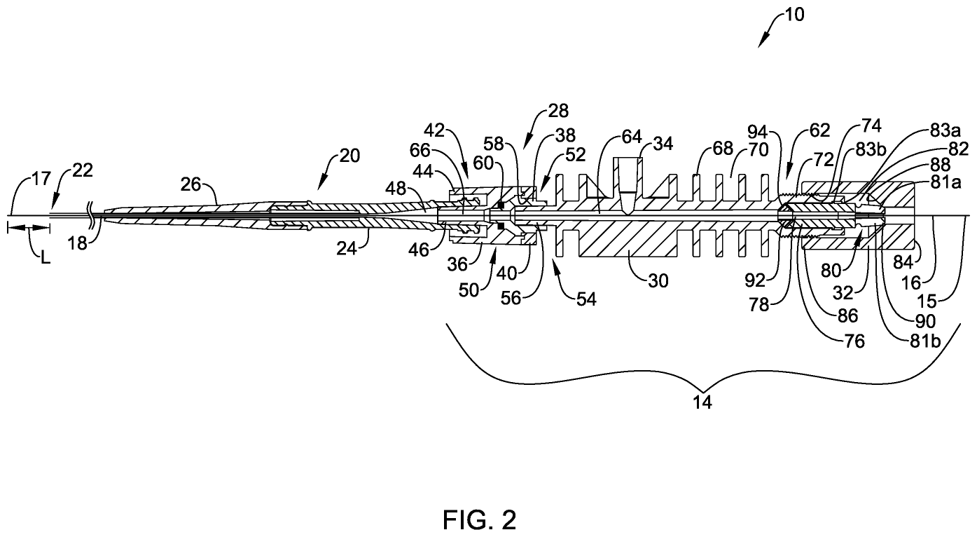

The details of the torque assembly 14 are illustrated in FIG. 2, which is a cross-sectional view of the illustrative catheter system 10 taken at line 2-2 of FIG. 1. Generally, the torque assembly 14 may be coupled to the guidewire 16 such that axial and/or rotational movement of the torque assembly 14 is translated to the guidewire 16. Further, the torque assembly 14 may be selectively coupled to the hub assembly 24 such that axial and/or rotational movement of the torque assembly 14 may be selectively translated to the hub assembly 24 and elongate shaft 18. As will be described in more detail herein, the torque assembly 14 may be coupled to the hub assembly 24 such that rotation of the torque assembly 14 is not necessarily translated to the hub assembly 24 and elongate shaft 18.

The rotating valve 28 may include a first connector portion 36, a second connector portion 38, and a coupling portion 40. The rotating valve 28 and the components thereof may be formed from a thermoplastic polymer. Some illustrative thermoplastic polymers may include, but are not limited to, polyethylene (PE), polypropylene (PP), polymethyl methacrylate (PMMA), polyvinyl chloride (PVC), polyamide, acrylonitrile butadiene styrene (ABS), and polycarbonate. Other materials, such as, but not limited to, other polymers, metals, metal alloys, ceramics, composites, etc. may also be used if so desired. A distal end portion 42 of the first connector portion 36 may be configured to be releasably coupled to a proximal end region of the hub assembly 24 of the elongate shaft 18. The distal end portion 42 of the first connector portion 36 may include an outer tubular member 44 and an inner tubular member 46. An annular opening may be defined between the outer tubular member 44 and the inner tubular member 46. The inner tubular member 46 may be sized and shaped to be disposed within a lumen 48 of the hub assembly 24. In some cases, the inner tubular member 46 may be configured to form a friction fit with the lumen 48 of the hub assembly 24. For example, an outer surface of the inner tubular member 46 may be configured to engage an inner surface of the hub assembly 24. It is contemplated that other releasable coupling mechanisms may be used, as desired, such as, but not limited to, snap fits, threaded engagements, bayonet style mechanisms, etc. When the inner tubular member 46 is coupled with the hub assembly 24, rotation of the first connector portion 36 may result in rotation of the hub assembly 24 and elongate shaft 18.

A proximal end portion 52 of the second connector portion 38 may be configured to be coupled to a distal end region 54 of the main body 30. The second connector portion 38 may be generally tubular and may define a lumen extending from the proximal end portion 52 to a distal end thereof. The lumen is coupled to a lumen of the inner tubular member 46 of the first connector portion 36 to define a lumen 66 extending from a proximal end of the rotating valve 28 to a distal end of the rotating valve 28. The lumen 66 may be configured to receive the guidewire 16 therethrough. The lumen of the second connector portion 28 may be sized and shaped to receive a distal tubular extension 56 of the main body 30. The distal tubular extension 56 may be secured to the second connector portion 38 such that movement of the main body 30 is translated to the second connector portion 38. For example, the main body 30 and the second connector portion 38 may be coupled such that rotation of the main body 30 results in a corresponding rotation of the second connector portion 38 and axial movement of the main body 30 results in a corresponding axial movement of the second connector portion 38. In some cases, the distal tubular extension 56 may be secured to the first connector portion 36 using any suitable technique, for example, by adhesive, friction fitting, mechanically fitting, chemically bonding, thermally bonding, heat shrink materials, molding, casting, welding (e.g., resistance or laser welding), soldering, brazing, the use of an outer sleeve or polymer layer to bond or connect the components, or the like, or combinations thereof.

The coupling portion 40 may be a generally annular ring configured to movably couple the first and second connector portions 36, 38. A proximal end region 50 of the first connector portion 36 may be rotatably coupled to a distal end of the coupling portion 40 and/or the second connector portion 38 while an inner surface 58 of the coupling portion 40 may be fixedly secured to the second connector portion 38. The second connector portion 38 and the coupling portion 40 may be coupled such that the second connector portion 38 moves with the coupling portion 40 and vice versa. In some instances, the first connector portion 36 may be configured to rotate with the coupling portion 40 and/or the second connector portion 38 while in other instances the first connector portion 36 may be configured to remain stationary while the coupling portion 40 and/or the second connector portion 38 is rotated. For example, a clinician may grip the first connector portion 36 to hold the first connector portion 36 in a stationary position while the coupling portion 40 and/or the second connector portion 38 is rotated. When the first connector portion 36 is held stationary, the rotation of the torque assembly 14 is not translated to the hub assembly 24 and elongate shaft 18. This may allow the clinician to torque the guidewire 16 independently of the elongate shaft 18. In some cases, an O-ring or other sealing member 60 may be positioned between the first connector portion 36 and the second connector portion 38. When the first connector portion 36 is free from the grip of a clinician, the first connector portion 36 may rotate with the second connector portion 38 such that rotation of the torque assembly 14 is translated to both the guidewire 16 and the elongate shaft 18 to allow for simultaneous torquing of both the guidewire 16 and the elongate shaft 18.

The main body 30 may extend proximally from the distal end region 54 to a proximal end region 62. The main body 30 be formed from a thermoplastic polymer. Some illustrative thermoplastic polymers may include, but are not limited to, polyethylene (PE), polypropylene (PP), polymethyl methacrylate (PMMA), polyvinyl chloride (PVC), polyamide, acrylonitrile butadiene styrene (ABS), and polycarbonate. Other materials, such as, but not limited to, other polymers, metals, metal alloys, ceramics, composites, etc. may also be used if so desired. A lumen 64 may extend from the proximal end to the distal end of the main body 30. A distal portion of the lumen 64 is configured to be in communication with the lumen 66 of the rotating valve 28. In some cases, the lumen 64 may have a first diameter adjacent to the proximal end region 62 of the main body 30 and a second, smaller, diameter adjacent to the distal end region 54 of the housing. A transition region 94 may extend between the first diameter and the second diameter. The lumen 64 may be configured to receive the guidewire 16 therethrough. The flush port 34 may be in fluid communication with the lumen 64 of the lumen 66 to allow for flushing of the guidewire 16 and/or elongate shaft 18 prior to use or for the introduction of therapeutics or devices. An outer surface of the main body 30 may be textured or otherwise include features to increase the gripability of the main body 30. In some examples, the textured outer surface may include a plurality of ridges 68 and a plurality of recesses 70. The recesses 70 may extend through less than an entire thickness of the main body 30 or may extend through less than half of a thickness of the main body 30. It is further contemplated that the textured surface may extend along less than entire length of the main body 30. An external surface of the proximal end region 62 of the main body 30 may include a plurality of threads 72. The plurality of external threads 72 may be configured to threadably engage mating internal threads 74 on the cap 32.

A collet 76 may be at least partially disposed within the lumen 64 of the main body 30 adjacent the proximal end region 62 thereof. The collet 76 may be formed from a thermoplastic polymer or metal. Some illustrative thermoplastic polymers may include, but are not limited to, polyethylene (PE), polypropylene (PP), polymethyl methacrylate (PMMA), polyvinyl chloride (PVC), polyamide, acrylonitrile butadiene styrene (ABS), and polycarbonate. Other materials, such as, but not limited to, other polymers, metals, metal alloys, ceramics, composites, etc. may also be used if so desired. In some cases, the collet 76 may be fixedly secured to the main body 30. The collet 76 may be secured to the main body 30 using any suitable technique, for example, by adhesive, friction fitting, mechanically fitting, chemically bonding, thermally bonding, heat shrink materials, molding, casting, welding (e.g., resistance or laser welding), soldering, brazing, the use of an outer sleeve or polymer layer to bond or connect the components, or the like, or combinations thereof. In some cases, the collet 76 and the main body 30 may include a mechanical interlock, such as, but not limited to, a pair of mating raised ridges 83a, 83b to prevent the collet 76 from disengaging from the main body 30.

The collet 76 may define a lumen 78 extending from a proximal end to a distal end thereof. The lumen 78 may be in communication with the lumen 64 of the main body 30. A proximal end region 80 of the collet 76 may be configured to selectively engage or grip an outer surface of the guidewire 16 to secure the guidewire 16 to the torque assembly 14 such that movement of the torque assembly 14 is translated to the guidewire 16. For example, the proximal end region 80 may include a pair of deflectable arms 81a, 81b (collectively, 81) configured to be deflected radially inwards to grip the guidewire 16. For example, as will be described in more detail herein, the cap 32 may be actuated to deflect the arms 81 to grip the guidewire 16. A hemostasis seal 92 may be positioned between a distal end of the collet 76 and the transition region 94 of the lumen 64 of the main body 30. The hemostasis seal 92 may configured to prevent blood and/or other fluids from exiting a proximal end of the torque assembly 14. The hemostasis seal 92 may be formed from a flexible rubber, silicone, elastomer, etc. such that the hemostasis seal 92 can form a fluid tight seal between the guidewire wire 16 and the main body 30.

The cap 32 may be disposed over the proximal end region 62 of the main body 30 and the proximal end region 80 of the collet 76. The cap 32 may define a lumen 82 extending from a proximal end 84 to a distal end 86 of the cap 32. The lumen 82 may have a first diameter adjacent to the proximal end 84 and a second diameter adjacent to the distal end 86. The second diameter may be larger than the first diameter. The lumen 82 may include a sloped transition region 88 positioned between the first diameter and the second diameter. The transition region 88 may provide a gradual transition from the first diameter to the second diameter. In some cases, the gradual transition may have a slope or angle that generally mates with a sloped outer surface 90 of a proximal end of the collet 76 to facilitate axial movement of the cap 32 relative to the collet 76. In a radially unbiased configuration, the arms 81 of the collet 76 may have an outer diameter greater than the first diameter of the lumen 82 of the cap 32 and greater than a proximal portion of the transition region 88.

As described herein, the cap 32 may include a plurality of internal threads 74 configured to threadably engage the external threads 72 of the main body 30. It is contemplated that the rotation of the cap 32 relative to the main body 30 may move the cap 32 axially along a longitudinal axis of the torque assembly 14. Rotation of the cap 32 in a first direction may move the cap 32 distally relative to the main body 30. This may bring an inner surface of the transition region 88 and/or a region of the lumen 78 having the first diameter into contact with the outer surface 90 of the proximal end of the collet 76. Further distal movement of the cap 32 may cause the transition region 88 to bias the arms 81 inward to grip the guidewire 16. When the collet 76 is locked to the guidewire 16, actuation of the main body 30 and/or cap 32 is translated to the guidewire 16. Rotation of the cap 32 in a second direction opposite the first direction may move the cap 32 proximally relative to the main body 30. This may allow the arms 81 of the proximal end region 80 to expand and release the guidewire 16. When the guidewire 16 is released from the collet 76, the guidewire 16 can be axially displaced along the longitudinal axis of the torque assembly 14 and elongate shaft 18 in a proximal and/or distal direction independent of the torque assembly 14 and/or elongate shaft 18. If a clinician desires to adjust the length L of the guidewire 16 extending distally from the distal end 22 of the elongate shaft 18, the cap 32 may be actuated to release biasing force of the arms 81 of the collet 76. The guidewire 16 may then be proximally retracted or distally advanced, as desired. When the desired length L of guidewire 16 extends distally beyond the distal end 22 of the elongate shaft 18, the cap 32 may be actuated to bias the arms 81 of the collet 76 radially inwards to grip the guidewire 16 and secure the guidewire 16 to the torque assembly 14.

In use, the guidewire 16 and the elongate shaft 18 may be torqued together or individually. To torque both the guidewire 16 and the elongate shaft 18 simultaneously, the torque assembly 14 may be locked with the hub assembly 24 of the elongate shaft 18 and the cap 32 locked to the guidewire 16, as shown in FIG. 1. For example, the rotating valve 28 may be coupled with the hub assembly 24. Also, the cap 32 may be actuated (e.g., distally advanced) to lock the torque assembly 14 to the guidewire 16 by deflecting the arms 81 of the collet 76 radially inward. With the torque assembly 14 locked to both the guidewire 16 and the elongate shaft 18, both the guidewire 16 and the elongate shaft 18 will move together. For example, the guidewire 16 and the elongate shaft 18 can be proximally retracted, distally advanced, and/or torqued together.

In some cases, it may be desirable to torque the guidewire 16 separate from the elongate shaft 18 (or vice versa). In one example, the torque assembly 14 may be locked with the hub assembly 24 of the elongate shaft 18 and the cap 32 locked to the guidewire 16. The clinician can hold the first connector portion 36 of the rotating valve 28 to prevent rotation of the first connector portion 36 and hence prevent rotation of the hub assembly 24 and elongate shaft 18. The main body 30 may then be rotated to torque the guidewire 16. For example, as described above, the second connector portion 38 may be coupled to the main body 30 such that movement of the main body 30 is translated to the second connector portion 38. When the first connector portion 36 is gripped by the clinician, rotations of the main body 30 may cause the second connector portion 38 to rotate while the first connector portion 36, the hub assembly 24, and elongate shaft 18 remain stationary.

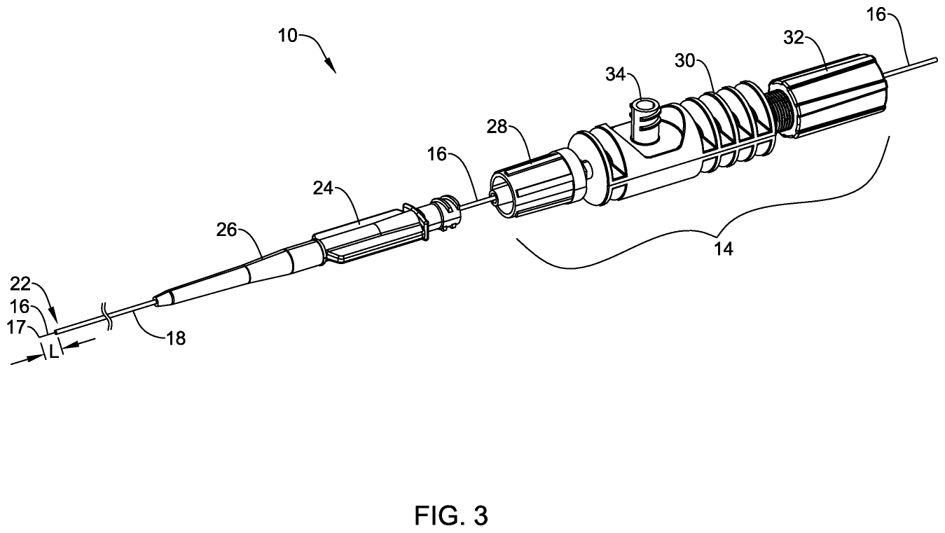

In another example, the torque assembly 14 may be unlocked from the hub assembly 24, as shown in FIG. 3 which illustrates a perspective view of the catheter system 10 with the torque assembly 14 unlocked from the hub assembly 24. With the collet 76 locked to the guidewire 16 and the torque assembly 14 unlocked from the hub assembly 24, the torque assembly 14 can be rotated and/or axially displaced to torque or axially displace the guidewire 16 without simultaneously moving the elongate shaft 18. In some cases, the torque assembly 14 may be unlocked from the hub assembly to adjust the length L of the guidewire 16 extending distally beyond the distal end 22 of the elongate shaft 18. For example, unlocking the torque assembly 14 from the hub assembly 24 may reduce the length L of guidewire 16 extending distally beyond the distal end 22 of the elongate shaft 18. If desired, the length L of the guidewire 16 extending distally beyond the distal end 22 of the elongate shaft 18 can be further adjusted by releasing the collet 76, moving the guidewire 18, and relocking the collet 76 to the guidewire 18.

The materials that can be used for the various components of the medical devices and/or systems (and/or other systems disclosed herein) and the various elements thereof disclosed herein may include those commonly associated with medical devices. For simplicity purposes, the following discussion makes reference to the catheter system 10. However, this is not intended to limit the devices and methods described herein, as the discussion may be applied to other elements, members, components, or devices disclosed herein, such as, but not limited to, the elongate shaft 18 and torque assembly 14, and/or elements or components thereof.

In some embodiments, the catheter system 10, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N0 and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of catheter system 10, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the catheter system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the catheter system 10 to achieve the same result.

In some embodiments, a degree of Magnetic Resonance Imaging (MRI) compatibility is imparted into the catheter system 10. For example, catheter system 10, and/or components or portions thereof, may be made of a material that does not substantially distort the image and create substantial artifacts (e.g., gaps in the image). Certain ferromagnetic materials, for example, may not be suitable because they may create artifacts in an MRI image. The catheter system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, an exterior surface of the catheter system 10 (including, for example, an exterior surface of the delivery system) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the outer sheath, or in embodiments without an outer sheath over portions of the delivery system, or other portions of the catheter system 10. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A catheter system, comprising:
a catheter having an elongate shaft extending from a proximal end to a distal end and defining a catheter lumen extending from the proximal end to the distal end;
a hub assembly coupled to the proximal end of the elongate shaft, the hub assembly including a hub assembly lumen in fluid communication with the catheter lumen;
a torque assembly releasably coupled to the hub assembly, the torque assembly including a torque assembly lumen in selective fluid communication with the hub assembly lumen; and
a guidewire, the guidewire co-axially disposed within the catheter lumen, hub assembly lumen, and torque assembly lumen;
wherein in a first configuration the torque assembly is configured to simultaneously torque the elongate shaft and the guidewire and in a second configuration the torque assembly is configured to torque the guidewire independent of the elongate shaft.

2. The catheter system of claim 1, wherein a length of the guidewire extending distally from the distal end of the elongate shaft is adjustable.

3. The catheter system of claim 1, wherein in the first configuration the torque assembly is coupled to the hub assembly.

4. The catheter system of claim 1, wherein in the second configuration the torque assembly is uncoupled from the hub assembly.

5. The catheter system of claim 1, wherein the torque assembly comprises a rotatable valve, a main body, a collet, and an actuatable cap.

6. The catheter system of claim 5, wherein the rotatable valve comprises a first coupling portion rotatably coupled to a second coupling portion, wherein the second coupling portion is configured to rotate independently of the first coupling portion when the first coupling portion is held in a fixed position.

7. The catheter system of claim 6, wherein when in the first configuration the torque assembly is configured to torque the guidewire independently of the catheter shaft when the first coupling portion is held in the fixed position.

8. The catheter system of claim 6, wherein a distal end region of the main body is fixedly secured to the second coupling portion.

9. The catheter system of claim 5, wherein the collet is configured to selectively lock to the guidewire.

10. The catheter system of claim 5, wherein the actuatable cap is actuated to selectively lock the collet to the guidewire.

11. The catheter system of claim 10, wherein the actuatable cap is configured to selectively bias a proximal end region of the collet radially inwards to lock the collet to the guidewire.

12. The catheter system of claim 9, wherein when the guidewire is unlocked from the collet, the guidewire is actuatable independent of the torque assembly.

13. The catheter system of claim 5, wherein the main body comprises a flush port in fluid communication with the torque assembly lumen.

14. The catheter system of claim 5, wherein the torque assembly further comprises a hemostasis seal.

15. The catheter system of claim 14, wherein the hemostasis seal is configured to form a fluid tight seal between the guidewire and the main body of the torque assembly.

16. A catheter system, comprising:
a catheter having an elongate shaft extending from a proximal end to a distal end and defining a catheter lumen extending from the proximal end to the distal end;
a hub assembly coupled to the proximal end of the elongate shaft, the hub assembly including a hub assembly lumen in fluid communication with the catheter lumen;
a torque assembly releasably coupled to the hub assembly, the torque assembly comprising a rotatable valve, a main body, a collet, and an actuatable cap and including a torque assembly lumen in selective fluid communication with the hub assembly lumen; and
a guidewire, the guidewire co-axially disposed within the catheter lumen, hub assembly lumen, and torque assembly lumen and selectively secured relative to the torque assembly;
wherein in a first configuration the torque assembly is configured to simultaneously torque the elongate shaft and the guidewire and in a second configuration the torque assembly is configured to torque the guidewire independent of the elongate shaft; and
wherein a length of the guidewire extending distally from the distal end of the elongate shaft is adjustable.

17. The catheter system of claim 16, wherein the torque assembly further comprises a hemostasis seal.

18. The catheter system of claim 16, wherein in the first configuration the torque assembly is coupled to the hub assembly and in the second configuration the torque assembly is uncoupled from the hub assembly.

19. The catheter system of claim 16, wherein the rotatable valve comprises a first coupling portion rotatably coupled to a second coupling portion, wherein the second coupling portion is configured to rotate independently of the first coupling portion when the first coupling portion is held in a fixed position and when in the first configuration the torque assembly is configured to torque the guidewire independently of the catheter shaft when the first coupling portion is held in the fixed position.

20. A catheter system, comprising:
a catheter having an elongate shaft extending from a proximal end to a distal end and defining a catheter lumen extending from the proximal end to the distal end;
a hub assembly coupled to the proximal end of the elongate shaft, the hub assembly including a hub assembly lumen in fluid communication with the catheter lumen;
a torque assembly releasably coupled to the hub assembly, the torque assembly comprising a rotatable valve including a first coupling portion rotatably coupled to a second coupling portion, a main body, a collet, and an actuatable cap and including a torque assembly lumen in selective fluid communication with the hub assembly lumen; and
a guidewire, the guidewire co-axially disposed within the catheter lumen, hub assembly lumen, and torque assembly lumen and selectively secured relative to the torque assembly;
wherein in a first configuration the torque assembly is coupled to the hub assembly and is configured to simultaneously torque the elongate shaft and the guidewire and in a second configuration the torque assembly is uncoupled from the hub assembly and is configured to torque the guidewire independent of the elongate shaft;
wherein a length of the guidewire extending distally from the distal end of the elongate shaft is adjustable; and
wherein when in the first configuration the torque assembly is configured to torque the guidewire independently of the catheter shaft when the first coupling portion is held in the fixed position.
